(19) United States
(12) Patent Application Publication
(10) Pub. No.: US 2025/0090461 A1
(43) Pub. Date: Mar. 20, 2025

(54) CELL-WALL BINDING PROTEIN SPECIFICALLY TARGETING CUTIBACTERIUM ACNES

(71) Applicant: MASSACHUSETTS INSTITUTE OF TECHNOLOGY, Cambridge, MA (US)

(72) Inventor: Boon Chong Goh, Singapore (SG)

(21) Appl. No.: 18/821,025

(22) Filed: Aug. 30, 2024

Related U.S. Application Data
(63) Continuation of application No. PCT/US2023/063698, filed on Mar. 3, 2023.
(60) Provisional application No. 63/316,173, filed on Mar. 3, 2022.

(51) Int. Cl. A61K 9/127; A61K 9/51; A61K 31/327; A61K 45/06; A61P 17/10; C12N 9/36

(52) U.S. Cl. CPC A61K 9/1271; A61K 9/5123; A61K 9/5153; A61K 31/327; A61K 45/06; A61P 17/10; C12N 9/2462; C12Y 302/01017

(57) ABSTRACT

The present invention relates to recombinant targeting peptides, compositions comprising said targeting peptides and methods of targeted delivery of therapeutics suitable for the control, improvement and/or treatment of acne. More particularly, the present invention provides engineered targeting peptides that bind specifically to the cell wall of Cutibacterium acnes, capable of providing a homing mechanism for transporting nanoparticles comprising therapeutics to C. acnes. Accordingly, the present invention also provides methods and compositions comprising the recombinant targeting peptides for localized delivery of therapeutics to C. acnes.

### CROSS REFERENCE TO RELATED APPLICATIONS

This application is a continuation application of PCT/US2023/063698 filed Mar. 3, 2023, which claims priority to and benefit of U.S. provisional patent application Ser. No. 63/316,173 filed Mar. 3, 2022, which is fully incorporated by reference and made a part hereof.

### REFERENCE TO SEQUENCE LISTING

The sequence listing submitted on Dec. 10, 2024 as an .XML file entitled “10480-021US1_ST26.xml” created on Dec. 4, 2024, and having a file size of 10,907 bytes is hereby incorporated by reference pursuant to 37 C.F.R. § 1.52(e)(5).

### FIELD OF THE INVENTION

The present invention relates to recombinant targeting peptides, compositions comprising said targeting peptides and methods of targeted delivery of therapeutics suitable for the control, improvement and/or treatment of acne. More particularly, the present invention provides engineered targeting peptides that bind specifically to the cell wall of Cutibacterium acnes, capable of providing a homing mechanism for transporting nanoparticles comprising therapeutics to C. acnes. Accordingly, the present invention also provides methods and compositions comprising the recombinant targeting peptides for localized delivery of therapeutics to C. acnes.

### BACKGROUND OF THE INVENTION

Acne, also known as acne vulgaris, is a chronic inflammatory disorder of the pilosebaceous unit. It is one of the most common skin diseases in the world, in which nearly all adolescents and adults may experience at some point in their lives. Besides causing long-term physical effects like scarring, acne can also severely impact a patient's psychological, social, and emotional well-being. For example, acne can lower a patient's self-esteem and confidence, which may consequently affect his/her social interaction, academic and work performance. In some cases, the distress caused by acne may even lead to depression and suicidal thoughts.

One of the causes of acne has been strongly linked to the bacteria Cutibacterium acnes, a rod-shaped, anaerobic Gram-positive commensal bacteria which lives on most healthy adult skin. The bacteria primarily reside deep within follicles and pores, and feeds on sebum and by-products from surrounding skin tissue. The role of C. acnes in causing acne is complex and likely involves multiple pathological processes and contributing factors. For instance, when a pore becomes blocked by cellular debris and sebum, this may cause an overgrowth of C. acnes, which irritates the pore lining and induces inflammation, leading to acne.

Bacterial control is a central part of acne treatment. Active ingredients like benzoyl peroxide and antibiotics are commonly used to reduce the bacterial population on human skin. While these actives are effective, they come with several undesirable side effects. A common side effect of benzoyl peroxide is dry skin as it is a harsh bleach-like chemical. An overreliance of antibiotics has already given rise to antibiotic-resistant bacteria on the skin. For example, C. acnes is increasingly becoming resistant to antibiotic therapy. Additionally, benzoyl peroxide and antibiotics kill bacteria indiscriminately, including the beneficial bacteria that form our skin microbiome. Prolonged usage of these actives will permanently disrupt the microbiome, thereby leading to more acne, rashes and other skin complications.

Therefore, there is a need to provide improved agents, compositions and methods of producing said agents and compositions thereof, for the management and treatment of acne that overcome, or at least ameliorate, one or more of the disadvantages described above.

### SUMMARY OF THE INVENTION

Disclosed herein are an isolated recombinant targeting peptide which specifically targets C. acnes, a recombinant DNA molecule and an expression vector encoding said targeting peptide, nanoparticles and compositions comprising said targeting peptide, and methods of producing said targeting peptides, nanoparticles and compositions thereof, suitable for use in the management, treatment or prophylaxis of acne. It would be understood that the invention may be used for cosmetic treatment or therapeutic treatment depending on the active agents loaded in the nanoparticles, and the circumstances or nature of the acne.

In a first aspect of the invention, there is provided a composition comprising: (i) a nanoparticle; (ii) a targeting peptide that binds to the cell wall of Cutibacterium acnes; and (iii) a cargo comprising one or more antimicrobial agents and/or anti-acne active agents, wherein said nanoparticle encapsulates the cargo, and said targeting peptide is a component of the surface of said nanoparticle.

In a second aspect of the invention, there is provided a use of the composition of the first aspect in the manufacture of a medicament for the treatment or prophylaxis of acne.

In a third aspect of the invention, there is provided a method of treatment or prophylaxis of acne, comprising administering an efficacious amount of a composition of the first aspect to a subject in need of such treatment.

In a fourth aspect of the invention, there is provided an isolated recombinant DNA molecule comprising a DNA sequence encoding a Cutibacterium acnes-targeting peptide disclosed in the first aspect.

In a fifth aspect of the invention, there is provided an expression vector comprising the recombinant DNA molecule of the fourth aspect.

In a sixth aspect of the invention, there is provided the use of an expression vector of the fifth aspect for the recombinant production of a Cutibacterium acnes-targeting peptide.

In a seventh aspect of the invention, there is provided an isolated recombinant Cutibacterium acnes-targeting peptide comprising an amino acid sequence that has at least 85%, at least 90%, at least 95% or 100% identity with the amino acid sequence set forth in SEQ ID NO: 3 or SEQ ID NO: 5 and having Cutibacterium acnes cell wall-binding activity.

In an eighth aspect of the invention, there is provided a method for the production of a recombinant Cutibacterium acnes-targeting peptide as disclosed in the first aspect, comprising the steps: (i) cultivating a eukaryotic or prokaryotic cell that has been transfected with a recombinant DNA molecule of the fourth aspect, or an expression vector of the fifth aspect in a cultivation medium, and
(ii) recovering the expressed recombinant Cutibacterium acnes-targeting peptides from the cell or the cultivation medium.

In a ninth aspect of the invention, there is provided a method for the production of a Cutibacterium acnes-targeting nanoparticle, the method comprising mixing a cargo comprising one or more antimicrobial agents and/or anti-acne active agents with Poly(D,L-lactide-co-glycolide), (PLGA), then combining the mixture with polyvinyl alcohol (PVA) and sonicating to form anionic nanoparticles; extracting the PLGA and cargo nanoparticles; mixing a Cutibacterium acnes-targeting peptide as defined in the first aspect with the PLGA and cargo nanoparticles until the nanoparticles are coated with the targeting peptide.

Alternatively, a Cutibacterium acnes-targeting lipid nanoparticle may be produced by a method comprising mixing a cargo comprising one or more antimicrobial agents and/or anti-acne active agents with lipids to form anionic liposome+cargo nanoparticles, mixing a Cutibacterium acnes-targeting peptide defined in the first aspect with the liposome and cargo nanoparticles until the nanoparticles are coated with targeting peptide. Preferably, the lipids are Dipalmitoylphosphatidylcholine (DPPC) and 1,2-Distearoyl-sn-glycero-3 phosphorylethanolamine (DSPE).

Advantageously, the nanoparticles and compositions of the present disclosure help preserve the human skin microbiome by selectively targeting and killing C. acnes only. More advantageously, the specificity of the engineered nanoparticles and compositions of the present disclosure also reduce the effective concentration and the amount of the antimicrobial agent and/or anti-acne active agent required, thereby minimizing unfavourable side effects that may be associated with said agents. These and other advantages of the present disclosure will become readily apparent to those skilled in this art from the following detailed description.

### BRIEF DESCRIPTION OF THE FIGURES

The accompanying drawings illustrate disclosed embodiments and serve to explain the principles of the disclosed embodiments. It is to be understood, however, that the drawings are designed for purposes of illustration only, and not as a definition of the limits of the invention.

FIG. 1 shows the binding spectrum of the SmartNovaC. Confocal microscopy images of SmartNovaC with Cutibacterium acnes 6919 (A1-3), C. acnes 11828 (B1-3), C. acnes 11827 (C1-3), C. acnes HC038-PAI (D1-3), Enterococcus faecalis OG1 RF (E1-3), Pseudomonas aeruginosa (F1-3), and Staphylococcus epidermidis (G1-3).

FIG. 2 shows the confocal microscopy images of NovaC with C. acnes 6919 (A1-3) and C. acnes 11828 (B1-3); DukeC2Rap with C. acnes 6919 (C1-3) and C. acnes 11828 (D1-3). White bars indicate 10 μm.

FIG. 3 shows a schematic conceptualization of SmartArrow. The SmartArrow constitutes of a lipid- or polymer-based nanoparticle that encapsulates/loads the anti-acne active ingredients, and the surface of the nanoparticle is coated with SmartNovaC.

FIG. 4 shows the Minimum Inhibitory Concentration (MIC) values of benzoyl peroxide (BPO) on Cutibacterium acnes. An OTC product containing 10% BPO was used in this experiment.

FIG. 5 shows a schematic of single emulsion technique.

FIG. 6 depicts the direct correlation between the input amount of BPO used experimentally and the amount of BPO loaded into PLGA nanoparticle as measured by mass spectroscopy. The input amount of PLGA was 5 mg.

FIG. 7 shows SmartNovaC coated on liposome and polymer nanoparticles. (A, C) The positively charged SmartNovaC, when coated on the surface of the nanoparticles, effectively reduces the net charge of the nanoparticles, as reflected by the zeta potential values. (B, D) The coating of the nanoparticles that was done using GFP-SmartNovaC can be quantified by measuring the fluorescence signal.

FIG. 8 depicts confocal microscopy images of SmartArrow nanoparticles loaded with nile red fluorescent dye and coated with SmartNovaC binding on C. acnes bacterial cells. The treated cells were imaged in (A) green channel, (B) red channel, (C) bright field. Image in (D) was generated by combining images from (A)-(C).

FIG. 9 shows the efficacy of BPO-loaded and SmartNovaC-coated SmartArrow nanoparticles. The number of colony forming unit (CFU) in each sample was determined by plating 10-fold serial dilution and compared to that of buffer-treated controls by a two-tailed Student's t-test with Welch's correction. *P<0.05; **P<0.01.

FIG. 10 shows the results of the predicted secondary structure location(s) in supernova using JPRED. JPRED, a secondary structure prediction web server, suggests residue 171 is a good starting residue to define the CBD of supernova.

FIG. 11 shows a photo of NovaC, visualized using software VMD. The dark helix (white arrow), which consists of 17 residues was removed to create SmartNovaC.

### DETAILED DESCRIPTION OF THE INVENTION

Bibliographic references mentioned in the present specification are for convenience listed in the form of a list of references and added at the end of the examples. The whole content of such bibliographic references is herein incorporated by reference but their mention in the specification does not imply that they form part of the common general knowledge.

### Definitions

For convenience, certain terms employed in the specification, examples and appended claims are collected here.

In general, technical, scientific and medical terminologies used herein has the same meaning as understood by those skilled in the art to which this invention belongs. Further, the following technical comments and definitions are provided. These definitions should in no way limit the scope of the present invention to those terms alone, but are put forth for a better understanding of the following description.

As used herein, “a” or “an” may mean one or more than one unless indicated to the contrary or otherwise evident from the context.

As used herein, “antimicrobial agent” refers to a natural or synthetic substance that kills or inhibits the growth of microorganisms such as bacteria, fungi and algae. Used in this context, an “antimicrobial agent” preferably kills or inhibits the growth of bacteria C. acnes.

As used herein, “anti-acne active agent” refers to any a natural or synthetic substance which may have a beneficial cosmetic effect and/or a beneficial therapeutic effect against the skin disease acne and its associated symptoms and presentations. Used in this context, an “anti-acne active agent” may, for example, reduce or control the number of acne blemishes, acne pimples, blackheads, and whiteheads, reduce or control sebum production, reduce, control or soothe the inflammation/swelling associated with acne (for example, reducing the redness appearance of the skin), and/or control the development of acne in a patient. As would be appreciated by a skilled artisan, some anti-acne active agent may provide a beneficial cosmetic effect only, while other anti-acne active agents may provide beneficial therapeutic effect or both.

As used herein, the term “cargo” refers to any compound/agent of interest that is intended to be delivered via a delivery system to a specific cellular destination to elicit a response. Used in this context, said cargo may be any compound or agent, such as an antimicrobial agent or an anti-acne active agent, intended to be transported by the delivery system of the present disclosure to where C. acnes reside, and released therein so that said cargo may interact with said bacteria to elicit a localised effect.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

As used herein, the terms “an efficacious amount” and “an effective amount” are used interchangeably and refer to an amount of the cargo and/or the composition comprising the cargo which is sufficient to effect the beneficial or desired results against C. acnes and/or acne. Used in this context, an effective amount of the composition of the present disclosure may result in, for example, killing and/or inhibition of C. acnes, reducing inflammation in or around the acne, beneficial cosmetic effect on said acne such as improvement in redness or appearance of acne-effected skin.

The term “functional fragment” refers to a portion of a protein that retains some or all of the activity or function (e.g., biological activity or function, such as enzymatic activity) of the full-length protein, such as, e.g., the ability to bind and/or interact with or modulate another protein or nucleic acid. The functional fragment can be any size, provided that the fragment retains, e.g., the ability to bind and interact with another protein or nucleic acid.

The term “variant”, as used herein, refers to an amino acid sequence that is altered by one or more amino acids of the non-variant reference sequence, but retains the ability to recognize its target and affect its function. For example, a targeting peptide variant is altered by one or more amino acids of the non-variant targeting peptide reference sequence, but retains the ability to recognize and bind to the cell wall of C. acnes. The variant may have “conservative” changes, wherein a substituted amino acid has similar structural or chemical properties (e.g., replacement of leucine with isoleucine). More rarely, a variant may have “non-conservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, DNASTAR® software (DNASTAR, Inc. Madison, Wisconsin, USA).

The terms “nucleotide” refer to naturally occurring ribonucleotide or deoxyribonucleotide monomers, as well as non-naturally occurring derivatives and analogs thereof. Nucleotides can include, for example, nucleotides comprising naturally occurring bases (e.g., adenosine, thymidine, guanosine, cytidine, uridine, inosine, deoxyadenosine, deoxythymidine, deoxyguanosine, or deoxycytidine) and nucleotides comprising modified bases known in the art. Accordingly, the term “polynucleotide” there relates in general to polyribonucleotides and polydeoxyribonucleotides, it being possible for these to be non-modified RNA or DNA or modified RNA or DNA.

As used herein, “peptide”, “polypeptide” and “protein” are used interchangeably to denote a polymer of at least two amino acids covalently linked by an amide bond, regardless of length or post-translational modification (e.g., glycosylation or phosphorylation). The term “protein” encompasses a naturally-occurring as well as artificial (e.g., engineered or variant) full-length protein as well as a functional fragment of the protein.

The term “recombinant” as used herein, means that a molecule (e.g., a nucleic acid or a polypeptide) has been artificially or synthetically (i.e., non-naturally) altered by human intervention. The alteration can be performed on the molecule within, or removed from, its natural environment or state.

A description of exemplary, non-limiting embodiments of the invention follows.

The present disclosure is based, in part, on the development of a recombinant peptide that has been engineered to advantageously bind to Cutibacterium acnes, a bacterial species closely linked to acne. In this regard, the inventors have successfully modified a native lysin derived from a bacteriophage named phage Supernova and improved its solubility and its binding affinity to the cell wall of C. acnes. Accordingly, the engineered targeting peptides disclosed herein are highly soluble and have an enhanced specificity to C. acnes compared to its native counterpart. The amino acid and polynucleotide sequences of native lysin and peptides isolated and developed therefrom are shown in Table 1.

TABLE 1
Amino acid and nucleotide sequences of the native and engineered
lysins of the present invention

| | |
|---|---|
| Native lysin: Supernova | MSSGVDLGTE NLYFQSMRYI PAAHHSAGSN |
| Amino acid sequence | KPVNRVVIHA TCPDVGFPSA SRKGRAVSTA |
| (Length: 303 amino acids) | NYFASPSSGG SAHYVCDIGE TVQCLSESTI |
| SEQ ID NO: 1 | AYTREQWLDP RVWPAVERAA IMCRQLCDKH |
| | GWHAPPNPHS LGIEICADGG SHASFRVPGH |
| | GVPKRKLSVA DLKAGRRGIC GHTDVTDAWH |
| | QSDHDDPGPW FPWDKFMAVV NGHGGGSSSE |
| | ELTVADVKAL HNQIKQLSAQ LSGSVNKLHH |
| | DVGVVQVQNG DLSKRVDALS WVKNPVTGKL |
| | WRTKDALWSV WYYVLECRSR IDRLESAVNG |
| | LKK |
| Native lysin: Supernova | ATGTCTTCTG GTGTAGATCT GGGTACCGAG |
| Nucleotide sequence | AACCTGTACT TCCAATCCAT GCGTTACATC |
| (Length: 912 bases) | CCGGCGGCGC ACCACAGCGC GGGCAGCAAC |
| SEQ ID NO: 2 | AAACCGGTTA ACCGTGTTGT TATCCACGCG |
| | ACTTGCCCGG ACGTTGGCTT CCCGAGCGCG |
| | AGCCGCAAAG GCCGTGCTGT TTCCACCGCG |
| | AACTACTTTG CTAGCCCGAG CAGCGGCGGC |
| | AGCGCTCACT ACGTGTGCGA TATCGGCGAA |
| | ACCGTGCAGT GCCTGAGCGA AAGCACCATC |
| | GGTTGGCACG CGCCGCCGAA CCCGCACTCT |
| | CTGGGTATCG AAATCTGCGC GGATGGCGGT |
| | AGCCACGCGT CTTTCCGTGT TCCGGGCCAC |
| | GCGTACACCC GTGAACAGTG GCTGGACCCG |
| | CGTGTTTGGC CGGCGGTTGA GCGTGCGGCG |
| | ATCATGTGCC GTCAGCTGTG CGACAAACAC |
| | GGTGTTCCGA AACGCAAACT GTCTGTTGCG |
| | GATCTGAAGG CTGGCCGCCG TGGCATCTGC |
| | GGTCACACCG ATGTAACCGA TGCTTGGCAT |
| | CAGTCTGATC ATGATGATCC AGGTCCGTGG |
| | TTCCCGTGGG ATAAATTTAT GGCGGTTGTT |
| | AACGGTCATG GTGGTGGTTC AAGCTCTGAA |
| | GAACTGACTG TGGCTGATGT TAAAGCACTG |
| | CACAATCAGA TTAAACAGTT AAGCGCACAA |
| | CTGAGCGGTT CTGTTAATAA ACTGCATCAC |
| | GATGTTGGTG TTGTTCAGGT TCAGAACGGT |
| | GATCTGAGCA AACGTGTTGA TGCTCTGTCC |
| | TGGGTTAAAA ATCCGGTTAC CGGTAAACTG |
| | TGGCGTACTA AAGACGCGCT GTGGAGTGTT |
| | TGGTATTACG TTCTGGAATG TCGTTCTCGT |
| | ATTGATCGTC TGGAAAGTGC GGTTAACGGT |
| | CTGAAAAAAT AA |
| CBD of native lysin: NovaC | FMAVVNGHGG GSSSEELTVA DVKALHNQIK |
| Amino acid sequence | QLSAQLSGSV NKLHHDVGVV QVQNGDLSKR |
| (Length: 108 amino acids) | VDALSWVKNP VTGKLWRTKD ALWSVWYYVL |
| SEQ ID NO: 3 | ECRSRIDRLE SAVNGLKK |
| CBD of native lysin: NovaC | CGTGGTTCCC GTGGGATAAA TTCATGGCGG |
| Nucleotide sequence | TTGTTAACGG TCACGGTGGT GGTTCTTCTT |
| (Length: 347 bases) | CTGAAGAACT GACCGTTGCT GATGTTAAAG |
| SEQ ID NO: 4 | CGCTGCACAA CCAGATCAAA CAGCTGTCTG |
| | CGCAGCTGAG CGGTTCTGTT AACAAACTGC |
| | ACCACGATGT TGGCGTTGTT CAGGTTCAGA |
| | ACGGTGATCT GAGCAAACGT GTTGATGCGC |
| | TGTCTTGGGT TAAAAACCCG GTTACCGGTA |
| | AACTGTGGCG TACCAAAGAT GCTCTGTGGT |
| | CTGTTTGGTA CTATGTTCTG GAATGCCGTA |
| | GCCGTATTGA TCGTCTGGAA AGCGCGGTTA |
| | ACGGTCTGAA AAAATAA |
| Engineered lysin: | MGGGSSSEEL TVADVKALHN QIKQLSAQLS |
| SmartNovaC | GSVNKLHHDV GVVQVQNGDL SKRVDALSWV |
| Amino acid sequence | KNPVTGKLWR TKDALWSVWY YVLECRSRID |
| (Length: 101 amino acids) | RLESAVNGLK K |
| SEQ ID NO: 5 | |
| Engineered lysin; | ATGGGTGGTG GTTCAAGCTC TGAAGAACTG |
| SmartNovaC | ACTGTGGCTG ATGTTAAAGC ACTGCACAAT |
| Nucleotide sequence | CAGATTAAAC AGTTAAGCGC ACAACTGAGC |
| (Length: 306 bases) | GGTTCTGTTA ATAAACTGCA TCACGATGTT |
| SEQ ID NO: 6 | GGTGTTGTTC AGGTTCAGAA CGGTGATCTG |
| | AGCAAACGTG TTGATGCTCT GTCCTGGGTT |
| | AAAAATCCGG TTACCGGTAA ACTGTGGCGT |
| | ACTAAAGACG CGCTGTGGAG TGTTTGGTAT |
| | TACGTTCTGG AATGTCGTTC TCGTATTGAT |
| | CGTCTGGAAA GTGCGGTTAA CGGTCTGAAA |
| | AAATAA |

As described herein, the modified targeting peptides of the present disclosure serve as a homing mechanism to deliver a cargo of interest to its designated location and, therefore, may be coupled to a cellular delivery system for a more precise, targeted, drug delivery system. Accordingly, the nanoparticles, compositions and methods of the present disclosure have been designed to advantageously target C. acnes only and not any other bacteria, thereby protecting the overall skin microbiome, without significant off-target effects.

In one aspect, there is provided a composition comprising: i) a nanoparticle; ii) a targeting peptide that binds to the cell wall of Cutibacterium acnes; and iii) a cargo comprising one or more antimicrobial agents and/or anti-acne active agents, wherein said nanoparticle encapsulates the cargo, and said targeting peptide is a component of the surface of said nanoparticle.

The compositions described herein enable the efficient, target-oriented delivery of a cargo of interest (for example, an antimicrobial agent) to C. acnes directly. In particular, nanoparticles and compositions disclosed herein may be coated with the targeting peptides to provide the enhanced selectivity for C. acnes. Both covalent and non-covalent methods of coating said peptides may be employed.

In some embodiments, the targeting peptide comprises the amino acid sequence set forth in SEQ ID NO: 3, a functional fragment or a variant thereof having Cutibacterium acnes cell wall-binding activity.

As those skilled in the art would appreciate, a protein's function is directly related to its structure and sequence, and that there is a positive relationship between sequence identity and function similarity. In this regard, methods of determining a protein sequence identity are known in the art. Therefore, the sequences of the targeting peptides disclosed herein may be sufficiently varied so long as the targeting peptides maintain their functionality and can exhibit the required activity (for example, the targeting peptide being able to bind to the cell wall of C. acnes).

Accordingly in some embodiments, the targeting peptide may comprise an amino acid sequence with at least 85%, at least 90%, at least 95%, at least 96%, at least 97%, at least 98%, at least 99% or 100% identity to the amino acid sequence set forth in SEQ ID NO. 3. In particular, the targeting peptide may consist of the amino acid sequence set forth in SEQ ID NO: 3.

In some embodiments, the targeting peptide may comprise an amino acid sequence with at least 85%, at least 90%, at least 95%, at least 96%, at least 97%, at least 98%, at least 99% or 100% identity to the amino acid sequence set forth in SEQ ID NO. 5. In some embodiments, the targeting peptide may consist of the amino acid sequence set forth in SEQ ID NO: 5.

In another aspect, there is provided an isolated recombinant Cutibacterium acnes-targeting peptide comprising an amino acid sequence that has at least 85%, at least 90%, at least 95% or 100% identity with the amino acid sequence set forth in SEQ ID NO: 3 or SEQ ID NO: 5 and having Cutibacterium acnes cell wall-binding activity.

As those skilled in the art would appreciate, a peptide may be encoded by a sequence of nucleotides, which is read in groups of three nucleotides, known as a codon. Accordingly in some embodiments, the targeting peptide may be encoded by a polynucleotide comprising a nucleic acid sequence that has, due to redundancy in the genetic code, at least 80%, at least 85%, at least 90%, at least 95%, at least 96%, at least 97%, at least 98%, at least 99% or 100% identity with the nucleic acid sequence set forth in SEQ ID NO: 4.

In some embodiments, the targeting peptide may be encoded by a polynucleotide comprising a nucleic acid sequence that has, due to redundancy in the genetic code, at least 80%, at least 85%, at least 90%, at least 95%, at least 96%, at least 97%, at least 98%, at least 99% or 100% identity with the nucleic acid sequence set forth in SEQ ID NO: 6.

In another aspect, there is provided an isolated recombinant DNA molecule comprising a DNA sequence encoding a Cutibacterium acnes-targeting peptide as disclosed herein. In some embodiments, the DNA sequence encoding the targeting peptide has, due to redundancy in the genetic code, at least 80%, at least 85%, at least 90%, at least 95%, at least 96%, at least 97%, at least 98%, at least 99% or 100% nucleic acid sequence identity to the nucleic acid sequence set forth in SEQ ID NO: 4 or SEQ ID NO: 6.

In a further aspect, there is provided an expression vector comprising the recombinant DNA molecule of the present disclosure. It would be appreciated that an expression vector is a construct designed for gene expression in cells and are typically used for the production of proteins. Methods of constructing expression vectors are well known in the art.

Accordingly in another aspect, there is provided a use of an expression vector disclosed herein for the recombinant production of a Cutibacterium acnes-targeting peptide.

In a further aspect, there is provided a method for the production of a recombinant Cutibacterium acnes-targeting peptide of the present disclosure, comprising the steps: (i) cultivating a eukaryotic or prokaryotic cell that has been transfected with a recombinant DNA molecule or an expression vector as disclosed herein in a cultivation medium, and (ii) recovering the expressed recombinant Cutibacterium acnes-targeting peptides from the cell or the cultivation medium.

As described herein, the compositions of the present disclosure may be loaded with any cargo which is desired to be delivered to and interact with C. acnes. For example, said cargo may exhibit anti-microbial properties, anti-acne properties, anti-inflammatory properties and/or exhibit a therapeutic effect against C. acnes. It would be appreciated by a skilled artisan that any molecule or agent that may provide a beneficial effect, whether therapeutic, cosmetic or otherwise, in the control, improvement and/or treatment of acne may be suitably selected as a cargo.

In some embodiments, the cargo may comprise one or more antimicrobial agents. For example, the antimicrobial agent may be a small molecule. Antimicrobial agents may include, but are not limited to, benzoyl peroxide, sulphur, azelaic acid, and antibiotics such as ozenoxacin, nadifloxacin, doxycycline, minocycline, azithromycin, erythromycin, clindamycin and dapsone. In some embodiments, the one or more antimicrobial agents may comprise benzoyl peroxide, azelaic acid, antibiotics such as erythromycin, clindamycin, dapsone and/or a combination thereof.

In some embodiments, the cargo may comprise one or more anti-acne active agents. Anti-acne active agents may include, but are not limited to, alpha hydroxy acids such as glycolic acid and lactic acid; beta hydroxy acids such as salicylic acid; retinoids such as adapalene, tretinoin, isotretinoin, tazarotene, alitretinoin, bexarotene, resorcinol, retinyl esters, retinaldehyde, retinal and retinol; flavonoids and/or vitamin derivatives such as niacinamide and resorcinol. In some embodiments, the cargo may comprise one or more antimicrobial agents and/or anti-acne active agents.

In some embodiments, the anti-acne active agent may comprise compounds and/or extracts that may have cosmetic effect in managing and/or improving acne, such as tea tree oil, propolis extract, green tea extract, rice extract, astringents, anti-inflammatory compounds or a mixture thereof.

Accordingly in some embodiments, the compositions disclosed herein may be suitable for use as a cosmetic product, based on the appropriate cargo selected.

To facilitate the delivery of the cargo to where the bacteria reside, the cargo may be encapsulated in a nanoparticle. It would be appreciated that nanoparticles have been utilised in the medical/pharmaceutical field as drug carriers by encapsulating or attaching therapeutic molecules and deliver them to target tissues more precisely with a controlled release.

In this regard, nanoparticles are typically submicron (<1 μm) colloidal particles which exhibit unique structural, chemical, mechanical, magnetic, electrical, and biological properties.

Nanoparticles may be made from biocompatible and biodegradable materials such as lipids, natural polymers (for example, gelatin, albumin, alginate, chitosan) or synthetic polymers (for example, polyvinyl alcohol, poly-L-lactic acid, polyethylene glycol, poly(lactic-co-glycolic acid polylactides, polyalkylcyanoacrylates etc.). In some embodiments, the nanoparticle comprises a lipid-based structure such as liposome or micelle. In some embodiments, the nanoparticle may comprise poly(lactic acid) (PLA); polyglycolic acid (PGA); Poly(D,L-lactide-co-glycolide) (PLGA); or 1,2-dimyristoyl-sn-glycero-3-phosphocholine (DMPC) and 1,2-dimyristoyl-sn-glycero-3-phosphorylglycerol sodium salt (DMPG), or Dipalmitoylphosphatidylcholine (DPPC) and 1,2-Distearoyl-sn-glycero-3-phosphorylethanolamine (DSPE).

It would be appreciated by those skilled in the art that the selection and/or preparation of a suitable nanoparticle is based on factors such as the biophysical and biochemical properties of the cargo of interest as well as the target location. For example, the cargo may be encapsulated by the nanoparticle via hydrophobic effect, electrostatic interaction and/or covalent conjugation, depending on the physicochemical characteristic of the cargo. As such, the preparation of a suitable nanoparticle may be adapted by a skilled artisan accordingly.

In some embodiments, the nanoparticle may have an anionic surface charge, a cationic surface charge, or a neutral surface charge. In particular, the nanoparticle may have an anionic surface charge.

Accordingly in a further aspect, there is provided a method for the production of a Cutibacterium acnes-targeting nanoparticle, the method comprising mixing a cargo comprising one or more antimicrobial agents and/or anti-acne active agents with Poly(D,L-lactide-co-glycolide), (PLGA), then combining the mixture with polyvinyl alcohol (PVA) and sonicating to form anionic nanoparticles; extracting the PLGA+cargo nanoparticles; mixing a Cutibacterium acnes-targeting peptide of the present disclosure with the PLGA+cargo nanoparticles until the nanoparticles are coated with said targeting peptide. In some embodiments, the PLGA and PVA are substituted by 1,2-dimyristoyl-sn-glycero-3-phosphocholine (DMPC) and 1,2-dimyristoyl-sn-glycero-3-phosphorylglycerol sodium salt (DMPG), or dipalmitoylphosphatidylcholine (DPPC) and 1,2-distearoyl-sn-glycero-3-phosphorylethanolamine (DSPE).

In some embodiments, the nanoparticles and compositions disclosed herein may further comprise a pharmaceutically acceptable carrier. Suitable pharmaceutical carriers typically will contain inert ingredients that do not interact with the agent or active ingredient. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's lactate and the like. Formulations can also include small amounts of substances that enhance the effectiveness of the active ingredient (e.g., emulsifying agents, solubilizing agents, pH buffering agents, wetting agents). Methods of encapsulation compositions (such as in a coating of hard gelatin or cyclodextran) are known in the art. For inhalation, the agent can be solubilized and loaded into a suitable dispenser for administration (e.g., an atomizer or nebulizer or pressurized aerosol dispenser).

For in vivo delivery, the nanoparticles and compositions disclosed herein can be delivered to a subject in need thereof by a variety of routes of administration including, for example, oral, dietary, topical, transdermal, or parenteral (e.g., intra-arterial, intravenous, intramuscular, subcutaneous injection, intradermal injection) routes of administration. Administration can be local or systemic. The actual dose and treatment regimen of said nanoparticles and/or compositions herein can be determined by a skilled physician, taking into account the nature of the condition being treated, and patient characteristics.

In some embodiments, the compositions as disclosed herein may preferably be formulated for topical application. Preferably, the topical formulation may be in the form of a liquid solution or mixture, dispersion, suspension, gel, lotion, emulsion, paste, cream, ointment, milk, pomade, spray or a medicated bandage, pad or mask. It would be appreciated that the methods to prepare topical formulations are known is the art and is based on standard principles and methods described in various pharmaceutical literature.

In another aspect, there is provided a use of a composition of the present disclosure in the manufacture of a medicament for the treatment or prophylaxis of acne. In some embodiments, the compositions and the medicament herein disclosed is for selectively killing and/or targeting Cutibacterium acnes on human skin. In some embodiments, the medicament is in the form of a cream, gel or ointment.

In a further aspect, there is provided a method of treatment or prophylaxis of acne, comprising administering an efficacious amount of a composition of the present disclosure to a subject in need of such treatment.

Unless otherwise indicated or otherwise evident from the context and understanding of one of ordinary skill in the art, values that are expressed as ranges can assume any specific value or subrange within the stated ranges in various embodiments, to the tenth of the unit of the lower limit of the range, unless the context clearly dictates otherwise. “About” in reference to a numerical value generally refers to a range of values that fall within +10%, in some embodiments ±5%, in some embodiments ±1%, in some embodiments ±0.5% of the value unless otherwise stated or otherwise evident from the context. In any embodiment in which a numerical value is prefaced by “about”, an embodiment in which the exact value is recited is provided. Where an embodiment in which a numerical value is not prefaced by “about” is provided, an embodiment in which the value is prefaced by “about” is also provided. Where a range is preceded by “about”, embodiments are provided in which “about” applies to the lower limit and to the upper limit of the range or to either the lower or the upper limit, unless the context clearly dictates otherwise. Where a phrase such as “at least”, “up to”, “no more than”, or similar phrases, precedes a series of numbers, it is to be understood that the phrase applies to each number in the list in various embodiments (it being understood that, depending on the context, 100% of a value, e.g., a value expressed as a percentage, may be an upper limit), unless the context clearly dictates otherwise. For example, “at least 1, 2, or 3” should be understood to mean “at least 1, at least 2, or at least 3” in various embodiments. It will also be understood that any and all reasonable lower limits and upper limits are expressly contemplated.

Having now generally described the invention, the same will be more readily understood through reference to the following examples which are provided by way of illustration, and are not intended to be limiting of the present invention.

### EXAMPLES

Standard molecular biology techniques known in the art and not specifically described were generally followed as described in Green and Sambrook, Molecular Cloning: A Laboratory Manual, Cold Springs Harbor Laboratory, New York (2012).

### Example 1: Generating C. acnes—Targeting Engineered Lysins

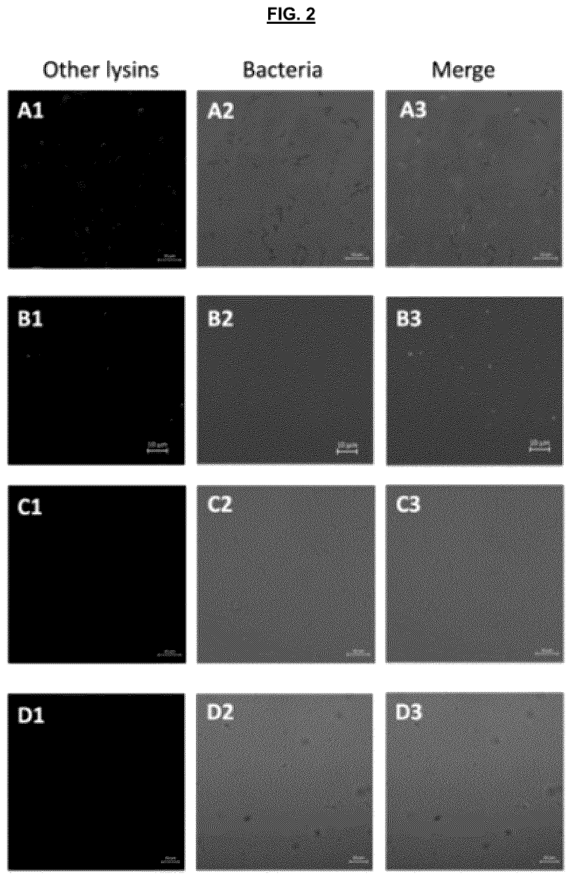

A native lysin named Supernova was selected as starting sequence template to generate engineered lysins. Supernova lysin was obtained from a bacteriophage, named phage Supernova, targeting Cutibacterium acnes. The C-terminal cell wall-binding domain (CBD) of Supernova, namely NovaC, was then determined to be from amino acid 171 to 287 based on results from BLAST and JPRED websites. However, the expression of NovaC is low and it shows very weak binding to C. acnes as shown in the confocal microscopy data (FIGS. 2A and 2B).

To improve the solubility of NovaC, the structure of NovaC was modeled using software I-TASSER and visualized using software VMD. From the in silico structure, the engineered lysin termed SmartNovaC was designed by truncating amino acid 2 to 18 of NovaC. This will remove an alpha helix that do not interact with the remaining protein.

The gene of native lysin Supernova (Genbank accession number ATN91960.1) was synthesized and cloned into pNIC28-Bsa4 plasmid. The engineered lysin genes (NovaC and SmartNovaC) were synthesized containing additional enhanced green fluorescent protein (EGFP) at the N-terminal and cloned into pET-22b (+) plasmid. All nucleotide sequences were codon-optimized to improve the efficiency of soluble expression in E. coli. The amino acid and nucleotide sequences of the native and engineered lysins are provided in Table 1.

To produce the recombinant lysins, the plasmids with the gene of interest are transformed to E. coli competent cells, and the proteins are overexpressed using IPTG induction. The proteins are purified by using immobilized metal affinity chromatography and size-exclusion chromatography.

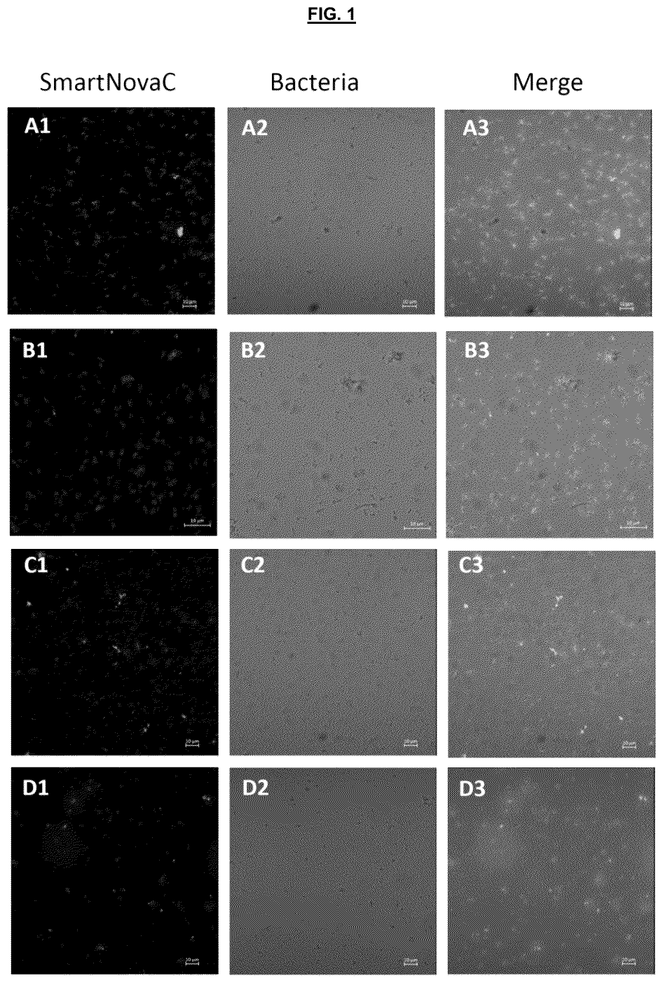

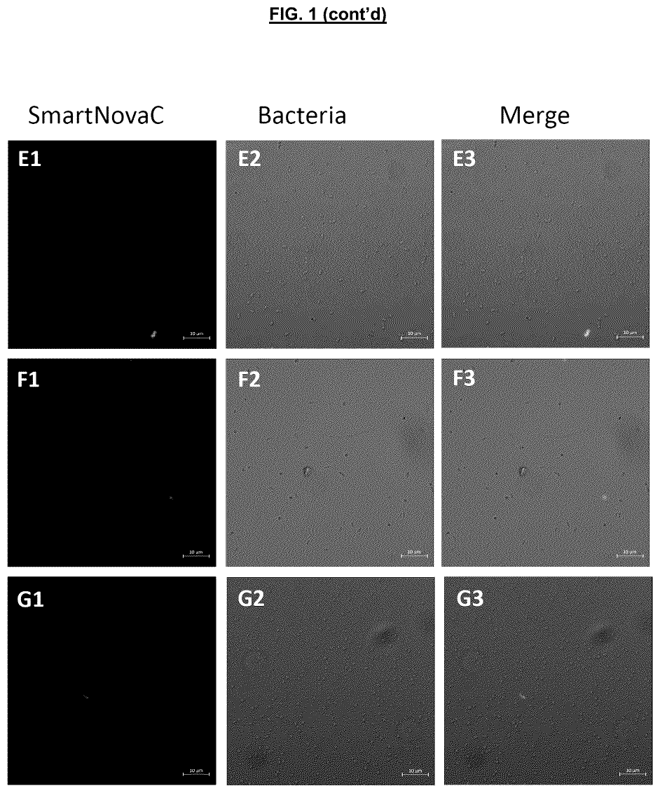

To check the binding spectrum of the engineered lysin SmartNovaC, 2.5 mg/ml of SmartNovaC was applied against four C. acnes strains, Enterococcus faecalis strain OG1 RF, Staphylococcus epidermidis strain PCI1200 and Pseudomonas aeruginosa strain PA14. Since SmartNovaC was cloned in a plasmid that would co-express the EGFP tag, the fluorescent lysin can be visualized using confocal microscopy. Green fluorescent SmartNovaC showed specific binding to all four strains of C. acnes (FIG. 1, A1-3, B1-3, C1-3, D1-3), while it could not bind to E. faecalis OG1RF (FIG. 1, E1-3), P. aeruginosa PA14 (FIG. 1, F1-3) or S. epidermidis PC11200 (FIG. 1, G1-3). This result clearly demonstrates the specific binding activity of SmartNovaC to C. acnes.

To further examine whether SmartNovaC is the only lysin that binds specifically to C. acnes, the binding of NovaC and another lysin DukeC2Rap that were synthesized with EGFP co-expressed were tested. DukeC2Rap lysin was obtained as CBD of Doucette lysin targeting Propionibacterium freudenreichii species, which is from the same genus as C. acnes. The nucleotide and amino acid sequences of the DukeC2Rap are provided below.

TABLE 2
Nucleotide and amino acid sequences
of the DukeC2Rap lysin

| | | | |
|---|---|---|---|
| DukeC2Rap lysin | MGGAASPMYA | DRLDGHQEYP | ETALFSLQQR |
| Amino acid | LGARFGELSG | KIDGLAEALK | QVQGGNGVDA |
| sequence | DAIRAAAAEG | AKAGVQAMID | TATVNLEAK |
| (Length: 89 | | | |
| amino acids) | | | |
| SEQ ID NO: 7 | | | |
| DukeC2Rap lysin | ATGGGTGGTG | CTGCTTCTCC | GATGTATGCT |
| Nucleotide | GATCGTCTGG | ATGGTCATCA | GGAATATCCG |
| sequence | GAAACCGCTC | TGTTTTCTCT | GCAGCAGCGT |
| (Length: | CTGGGCGCGC | GTTTCGGTGA | ACTGAGCGGC |
| 270 bases) | AAAATCGACG | GCCTGGCGGA | AGCGCTGAAA |
| SEQ ID NO: 8 | CAGGTGCAGG | GCGGCAACGG | CGTTGATGCG |
| | GATGCGATCC | GTGCGGCGGC | GGCGGAAGGT |
| | GCGAAAGCGG | GCGTTCAGGC | GATGATCGAT |
| | ACCGCGACCG | TTAACCTGGA | AGCGAAATAA |

NovaC and DukeC2Rap at concentrations of 2.5 mg/ml and 1.3 mg/ml, respectively, were tested for binding against two C. acnes strains. NovaC, although demonstrating some binding to C. acnes (FIG. 2, A1-3, B1-3), produced a binding signal that was much weaker than that of SmartNovaC (FIG. 1, A1-3, B1-3, C1-3, D1-3). This strongly suggests that the engineered SmartNovaC lysin has enhanced binding specificity to C. acnes. DukeC2Rap did not exhibit any binding to C. acnes (FIG. 2, C1-3, D1-3). Thus, among the different lysins tested, only the engineered lysin SmartNovaC possesses the specific binding to C. acnes species.

### Example 2: Preparation of Therapeutic Composition

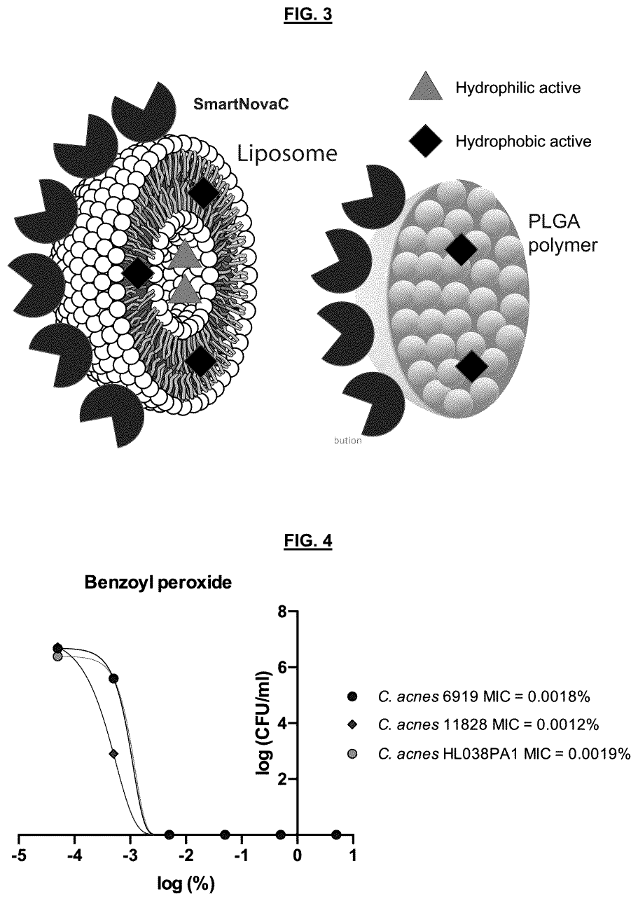

As SmartNovaC itself does not kill the bacteria directly, SmartNovaC needs to be combined with antibacterial agents. For example, to apply this technology to develop anti-acne products, SmartNovaC can be paired with anti-acne active ingredients such as benzoyl peroxide (BPO). As a result, a novel SmartArrow system was created, as illustrated in FIG. 3, by loading the active ingredients in a lipid/polymer-based nanoparticle, and coating the surface of the nanoparticle with SmartNovaC to selectivity target the antibacterial agents to C. acnes.

### Preparation of Anionic Liposome

An embodiment of the structure of a composition of the invention is illustrated in FIG. 3. Exemplary anionic liposomes were made from 1,2-dimyristoyl-sn-glycero-3-phosphocholine (DMPC) and 1,2-dimyristoyl-sn-glycero-3-phosphorylglycerol sodium salt (DMPG) at a 10:1 molar ratio. The size and stability of the liposome can be optimized by modifying the molar ratio, using different lipid types, and with addition of cholesterol, polyethylene glycol (PEG) and other additives that can change the behavior of the liposome.

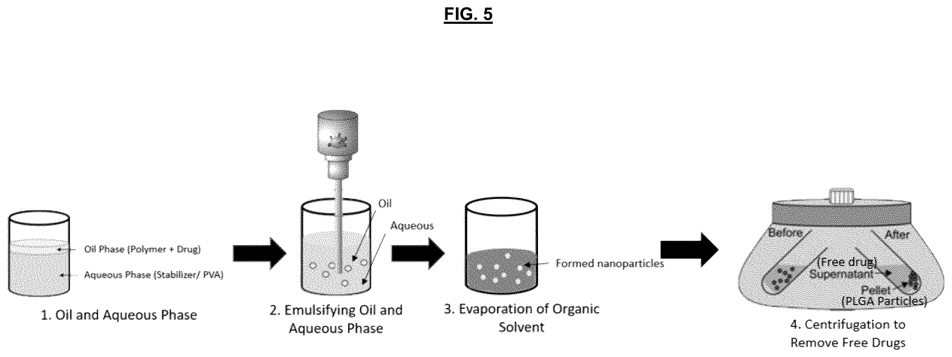

The advantage of SmartArrow is that it will deliver the active ingredients to where the bacteria reside. As a result, a much lower dosage of bioactive can be loaded in SmartArrow to achieve high killing of the bacteria compared to untargeted liposomes. As little as 0.002% BPO can achieve total killing of C. acnes (FIG. 4). Considering the BPO concentrations found in the retail market ranges from 2.5%-10%, which is ˜1000-fold higher than what is needed to kill the bacteria, it is not surprising to find most users of these retail products routinely suffer several side effects such as dry peeling skin. By using the SmartArrow approach, we can conservatively load 0.02% BPO into a liposome, which is 100× less than the retail products, to achieve total bacterial killing and reducing the side effects to a minimum.

### Preparation of PLGA+BPO Nanoparticles

To load BPO into a nanoparticle, Poly(D,L-lactide-co-glycolide), (PLGA), was chosen because it is known to form nanoparticles with excellent loading of hydrophobic compounds, such as BPO.

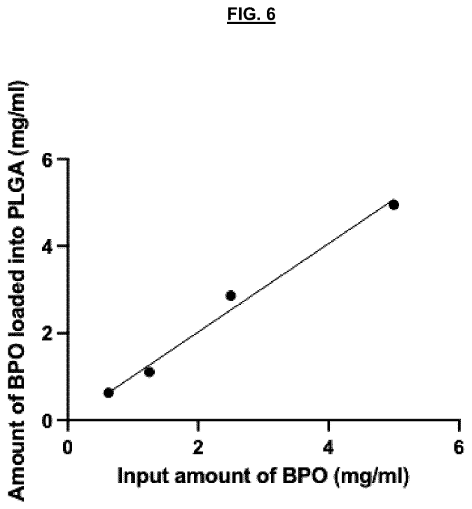

The BPO-containing PLGA particles were prepared using the single emulsion technique as previously described (Jain R A. 2000. Biomaterials 21: 2475-2490). Briefly, 5 g PLGA and 5 g BPO were each dissolved in 500 μl of chloroform. The PLGA and BPO were then added into 5 ml of 1% Polyvinyl alcohol (PVA) and the mixture was immediately sonicated at 23% amplitude for 5 minutes. Uniform, emulsified nanoparticles were formed after sonicating the oil phase (containing PLGA and BPO) and the aqueous phase (containing PVA) as shown in FIG. 5. To extract and remove the organic solvent, the mixture containing emulsified PLGA+BPO nanoparticles was left stirring at 700 rpm at room temperature for 6 hours in a fume hood. To purify for PLGA+BPO nanoparticles, the mixture was centrifuged at 6,000 rpm for 15 minutes to obtain a pellet containing the nanoparticles. The pellet was re-suspended with 2 ml of ultrapure water and spun down to remove all free BPO. The final pellet of purified PLGA+BPO nanoparticles was re-suspended with 2 ml of ultrapure water and stored at 4° C. until use.

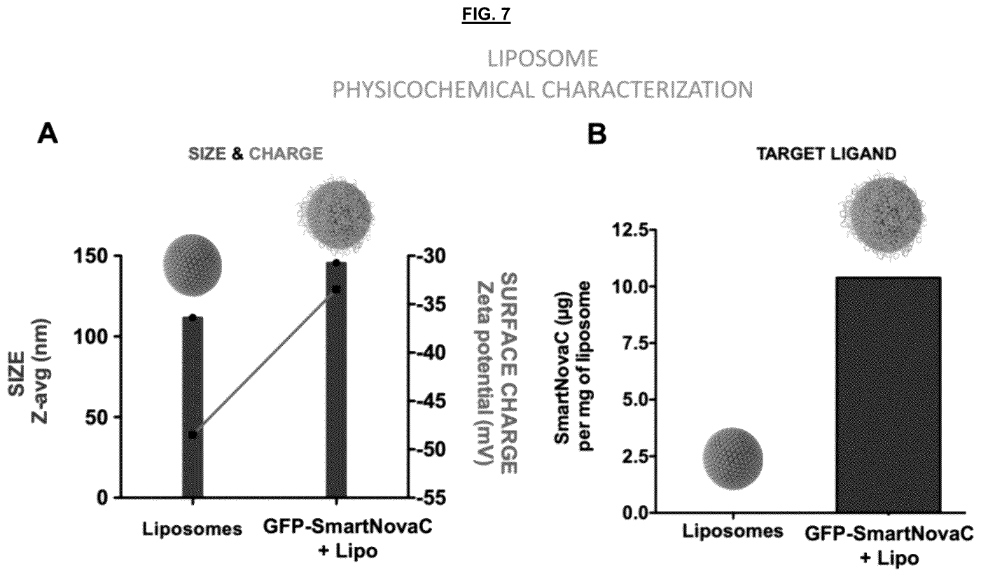

The PLGA+BPO nanoparticles were characterized using dynamic light scattering (DLS) and mass spectrometry. DLS measured the nanoparticles to have an average size of 150 nm with a zeta potential of −18 mV. The amount of BPO loaded in PLGA particles was quantified using mass spectroscopy, as shown in FIG. 6. We have shown that we can efficiently load up to 5 mg of BPO in 5 mg of PLGA particles. For the SmartArrow application of the invention, much less will likely be loaded.

### Preparation of DPPC+DSPE+BPO Nanoparticles

The BPO-containing DPPC+DSPE nanoparticles were prepared using 4 mg of DPPC and 1 mg of DSPE with 1 mg of BPO. Briefly, 4+1 mg DPPC+DSPE lipids and 1 mg BPO were dissolved in 500 μl of chloroform, respectively. The DPPC+DSPE lipids and BPO were then added into 4 ml of chloroform and the mixture was left in the fume hood overnight to allow evaporation of chloroform. The next day, 5 ml of 1× phosphate buffer saline (PBS) was added to rehydrate the dried lipid films. The solution was sonicated at 70° C. for 5 minutes for three rounds. The mixture will undergo a 400 nm extruder step as a filtration step and to form uniform nanoparticles. The filtered sample will undergo tangential flow filtration (TFF) using ultrapure water to separate the free BPO from the BPO-loaded nanoparticles. The final product was stored at 4° C. until use.

### Coating of SmartNovaC on Anionic Liposome and PLGA Nanoparticles Via Non-Covalent Methods.

The anionic liposome and PLGA nanoparticles were coated with the positively-charged SmartNovaC targeting peptide (both GFP-fused and free forms) using charge-based binding. The coating was done by mixing SmartNovaC peptide and the liposome/PLGA nanoparticles at a ratio of 2:1, and vortexing the mixture for 2 hours. To purify the protein-bound nanoparticles, the well-vortexed mixture was centrifuged at 6,000×g for 15 minutes. The pellet was re-suspended with 300 μl of ultrapure water and spun down via centrifugation to remove all unbound proteins. The final pellet was re-suspended with 100 μl of 1× phosphate buffer saline (PBS) and stored at 4° C. until use.

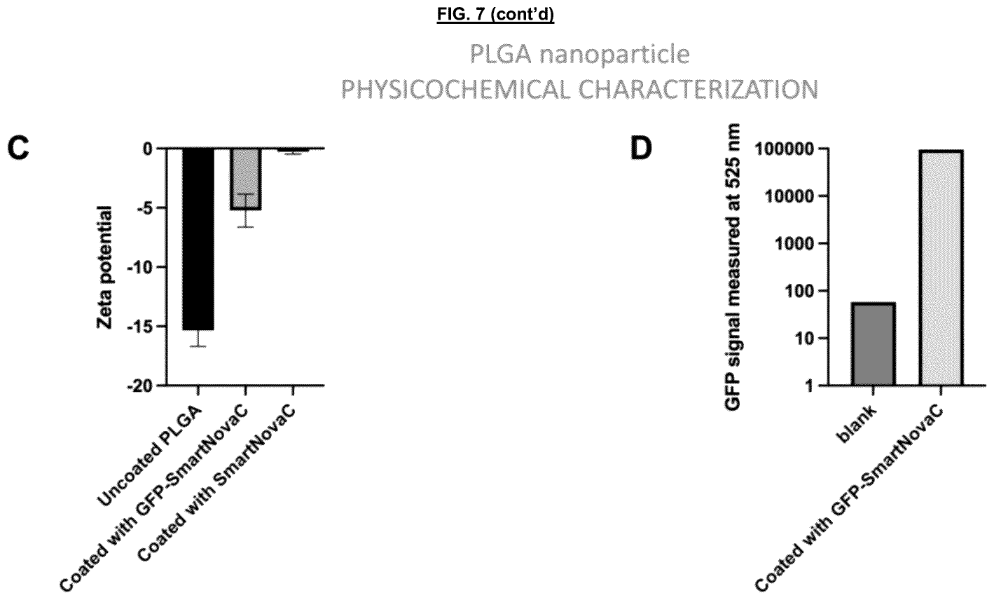

The resulting particles were characterized using dynamic light scattering (DLS). The SmartNovaC-coated liposome increased in size and decreased in zeta potential (FIG. 7A). This is consistent with the fact that positively-charged SmartNovaC coated on the anionic liposome will reduce the overall charge of the particle. Similarly, FIG. 7C shows a decrease in zeta potential of the PLGA nanoparticles upon SmartNovaC coating. FIGS. 7B and 7D show an increase in GFP fluorescence signal, thus the GFP-fused SmartNovaC was bound and coated onto the nanoparticles.

The coating approach shown in FIG. 7 was based on the principle of charge-based electrostatic interactions, but a more directional protein-nanoparticle bioconjugation can also be done to ensure SmartNovaC retain its selective binding on C. acnes.

### Coating of SmartNovaC on Lipid- or Polymer-Based Nanoparticles Via Site-Directed Covalent Conjugation.

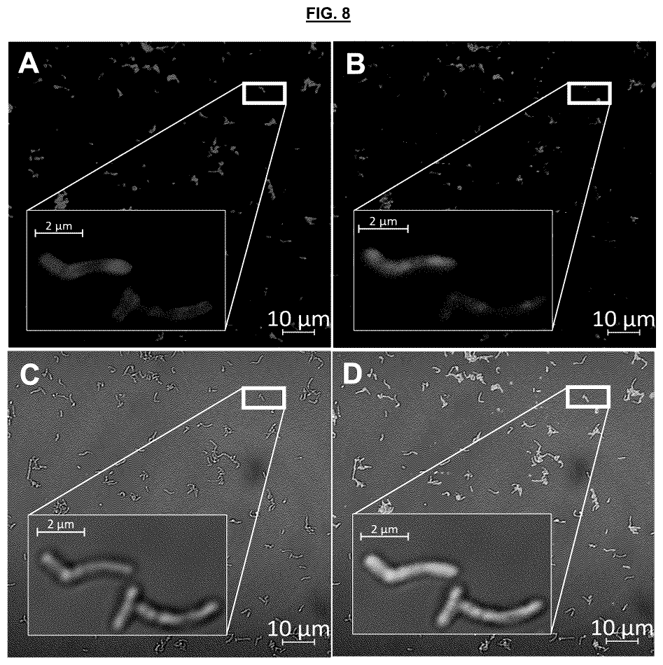

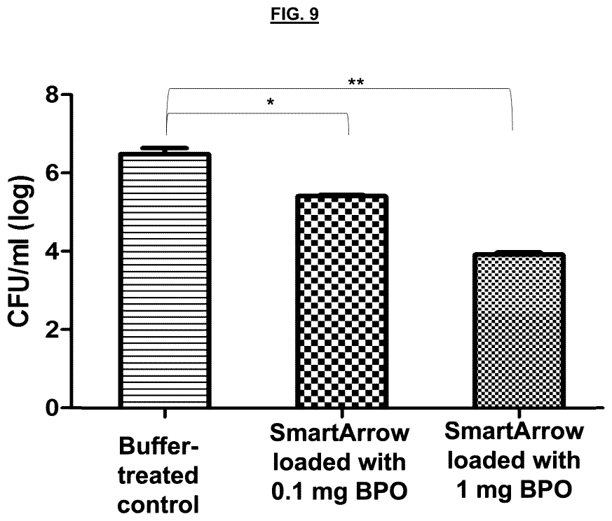

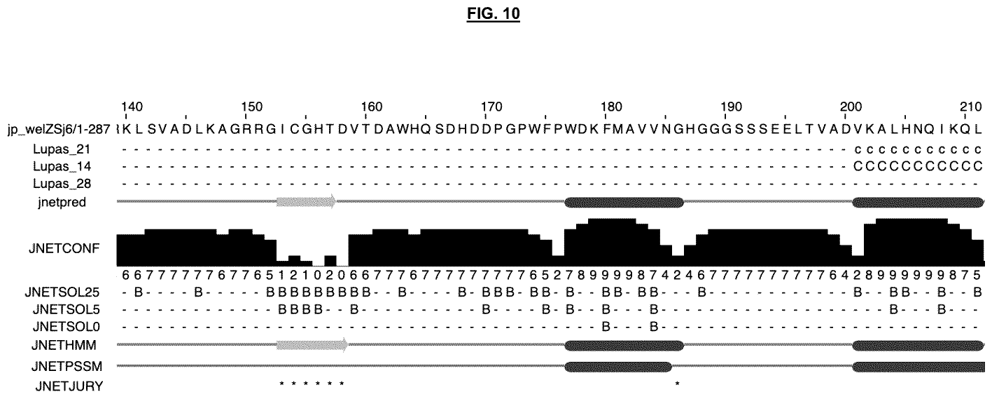

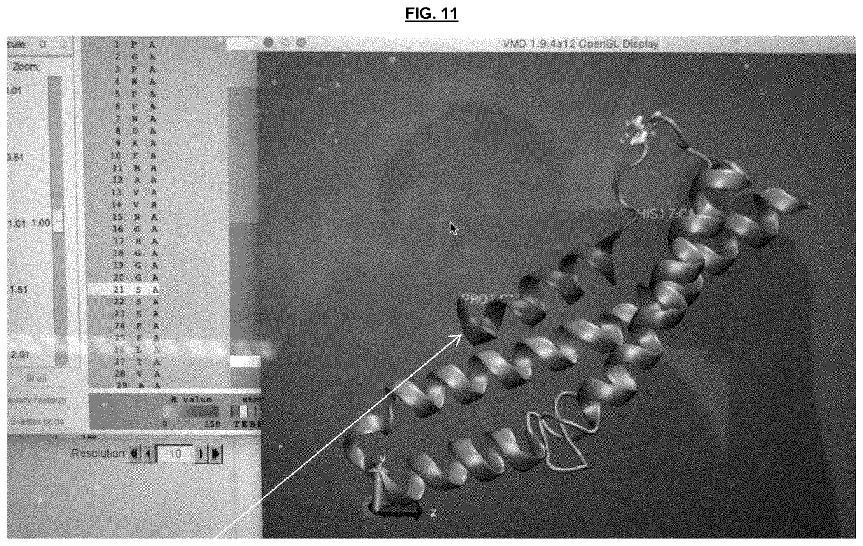

The thiol-maleimide reaction was used for the conjugation where the thiol group is found in SmartNovaC protein and the maleimide group is covalently linked to the lipid (e.g. DSPE and DPPC lipids) or polymer (e.g. PLGA). In a nanoparticle, only 20-30% of the lipid/polymer substrates contain the maleimide group to avoid overcrowding of conjugated protein, which may affect its targeting performance. According to DLS, the sizes of the nanoparticles when loaded with BPO and coated with conjugated SmartNovaC are in the range of 150-400 nm. FIG. 8 shows confocal microscopy images, in greyscale, of the SmartNovaC-coated, nile red-loaded polymeric nanoparticles binding to C. acnes. Both GFP (FIG. 8A) and nile red (FIG. 8B) signals overlap in the merged image FIG. 8D, demonstrating nanoparticle binding to the bacterial cells. FIG. 9 shows the BPO-loaded SmartArrow successfully exhibited bacterial killing, namely 90% of bacteria at 0.01% BPO and 99% bacteria at 0.1% BPO.

### SUMMARY

Presented herein is SmartNovaC, a 11 kDa cell-wall binding protein that is derived from a bacteriophage lysin. It has been conclusively shown that SmartNovaC can specifically bind to various strains of Cutibacterium acnes (C. acnes) and does not bind to the other bacteria. A delivery system comprising SmartNovaC and active ingredients like benzoyl peroxide would enable selective targeting and killing of C. acnes, thus respecting the skin microbiome. By incorporating SmartNovaC in the product formulation, it is estimated that the effective concentration of the active ingredient may be able to be reduced by 100× compared to the existing anti-acne products in the market.

The SmartArrow targeted delivery system involves loading anti-acne active ingredients into polymer/lipid-based nanoparticles and coating the surface of the nanoparticles with SmartNovaC peptide. Both covalent and non-covalent methods may be used for the coating using SmartNovaC. Accordingly, SmartArrow can be advantageously used in the skincare industry as a targeted delivery system for localized cosmetic or therapeutic acne treatment.

### REFERENCES

Jain R A. 2000. The manufacturing techniques of various drug loaded biodegradable poly(lactide-co-glycolide) (PLGA) devices. Biomaterials 21: 2475-2490.
Phage supernova genome: worldwideweb.ncbi.nlm.nih.gov/nuccore/MF91953

1. A composition comprising:
i) a nanoparticle;
ii) a targeting peptide that binds to the cell wall of Cutibacterium acnes; and
iii) a cargo comprising one or more antimicrobial agents and/or anti-acne active agents,
wherein said nanoparticle encapsulates the cargo, and said targeting peptide is a component of the surface of said nanoparticle.

2-24. (canceled)

25. The composition of claim 1, wherein the targeting peptide comprises an amino acid sequence that has at least 90%, at least 95% or at least 100% identity with the amino acid sequence:
(SEQ ID NO: 3)
MPGPWFPWDKFMAVVNGHGGGSSSEELTVADVKALHNQIKQLSAQLSGSV
NKLHHDVGVVQVQNGDLSKRVDALSWVKNPVTGKLWRTKDALWSVWYYVL
ECRSRIDRLESAVNGLKK,
or a functional fragment or variant thereof having Cutibacterium acnes cell wall-binding activity.

26. The composition of claim 25, wherein the targeting peptide comprises the amino acid sequence:
(SEQ ID NO: 3)
MPGPWFPWDKFMAVVNGHGGGSSSEELTVADVKALHNQIKQLSAQLSGSV
NKLHHDVGVVQVQNGDLSKRVDALSWVKNPVTGKLWRTKDALWSVWYYVL
ECRSRIDRLESAVNGLKK,
or a functional fragment or variant thereof having Cutibacterium acnes cell wall-binding activity.

27. The composition of claim 26, wherein the targeting peptide comprises an amino acid sequence that has at least 90%, at least 95% or 100% identity with the amino acid sequence;
(SEQ ID NO: 5)
MGGGSSSEELTVADVKALHNQIKQLSAQLSGSVNKLHHDVGVVQVQNGDL
SKRVDALSWVKNPVTGKLWRTKDALWSVWYYVLECRSRIDRLESAVNGLK
K.

28. The composition of claim 27, wherein the targeting peptide comprises the amino acid sequence:
(SEQ ID NO: 5)
MGGGSSSEELTVADVKALHNQIKQLSAQLSGSVNKLHHDVGVVQVQNGD
LSKRVDALSWVKNPVTGKLWRTKDALWSVWYYVLECRSRIDRLESAVNG
LKK.

29. The composition of claim 1, wherein the antimicrobial agent is a small molecule.

30. The composition of claim 1, wherein the one or more antimicrobial agents comprises benzoyl peroxide, azelaic acid, erythromycin, clindamycin, dapsone and/or a combination thereof.

31. The composition of claim 1, wherein said nanoparticle is selected from the group consisting of liposome, micelle, other lipid-based nanoparticle and other polymer-based nanoparticles.

32. The composition of claim 31, wherein the nanoparticle has an anionic surface charge.

33. The composition of claim 32, wherein the nanoparticle comprises:
i) Poly(D,L-lactide-co-glycolide) (PLGA), poly(lactic acid) (PLA) or polyglycolic acid (PGA); or
ii) 1,2-dimyristoyl-sn-glycero-3-phosphocholine (DMPC) and 1,2-dimyristoyl-sn-glycero-3-phosphorylglycerol sodium salt (DMPG); or
iii) Dipalmitoylphosphatidylcholine (DPPC) and 1,2-Distearoyl-sn-glycero-3 phosphorylethanolamine (DSPE).

34. The composition of claim 1, wherein the cargo comprises anti-acne active agents selected from:
i) alpha hydroxy acids, such as glycolic acid and lactic acid, and/or beta hydroxy acids, such as salicylic acid; and/or
ii) retinoids, such as adapalene, Isotretinoin, Tazarotene, retinal and retinol; and/or
iii) flavonoids and/or vitamin derivatives.

35. The composition of claim 1, further comprising a pharmaceutically acceptable carrier.

36. A method of treatment or prophylaxis of acne, comprising administering an efficacious amount of a composition of claim 1 to a subject in need of such treatment.

37. The method of claim 36, wherein the cargo comprises anti-acne active agents selected from:
i) alpha hydroxy acids, such as glycolic acid and lactic acid, and/or beta hydroxy acids, such as salicylic acid; and/or
ii) retinoids, such as adapalene, Isotretinoin, Tazarotene, retinal and retinol; and/or
iii) flavonoids and/or vitamin derivatives.

38. The method of claim 36, wherein the cargo comprises one or more antimicrobial agents.

39. The method of claim 38, wherein the antimicrobial agent is selected from benzoyl peroxide, sulphur, azelaic acid, erythromycin, clindamycin, dapsone, or a combination thereof.

40. The method of claim 39, wherein the antimicrobial agent is benzoyl peroxide administered at a load of 0.05%.

41. The method of claim 36, wherein the targeting peptide comprises an amino acid sequence that has at least 90%, at least 95% or 100% identity with the amino acid sequence;
(SEQ ID NO: 5)
MGGGSSSEELTVADVKALHNQIKQLSAQLSGSVNKLHHDVGVVQVQNGD
LSKRVDALSWVKNPVTGKLWRTKDALWSVWYYVLECRSRIDRLESAVNG
LKK.

42. The method of claim 36, wherein said nanoparticle is selected from the group consisting of liposome, micelle, other lipid-based nanoparticle and other polymer-based nanoparticles.

43. An isolated recombinant Cutibacterium acnes-targeting peptide comprising an amino acid sequence that has at least 90%, at least 95% or 100% identity with the amino acid sequence set forth in SEQ ID NO: 3 or SEQ ID NO: 5 and having Cutibacterium acnes cell wall-binding activity.


SEQUENCE LISTING (8 sequences)

SEQ ID NO: 1 - protein, 303 aa, unidentified
  source 1..303: note = Caudovirales siphoviridae family (formerly)
MSSGVDLGTENLYFQSMRYIPAAHHSAGSNKPVNRVVIHATCPDVGFPSASRKGRAVSTANYFASPSSGGSAHYVCDIGETVQCLSESTIGWHAPPNPHSLGIEICADGGSHASFRVPGHAYTREQWLDPRVWPAVERAAIMCRQLCDKHGVPKRKLSVADLKAGRRGICGHTDVTDAWHQSDHDDPGPWFPWDKFMAVVNGHGGGSSSEELTVADVKALHNQIKQLSAQLSGSVNKLHHDVGVVQVQNGDLSKRVDALSWVKNPVTGKLWRTKDALWSVWYYVLECRSRIDRLESAVNGLKK

SEQ ID NO: 2 - DNA, 912 bp, unidentified
  source 1..912: mol_type = genomic DNA; note = Caudovirales siphoviridae family (formerly)
atgtcttctggtgtagatctgggtaccgagaacctgtacttccaatccatgcgttacatcccggcggcgcaccacagcgcgggcagcaacaaaccggttaaccgtgttgttatccacgcgacttgcccggacgttggcttcccgagcgcgagccgcaaaggccgtgctgtttccaccgcgaactactttgctagcccgagcagcggcggcagcgctcactacgtgtgcgatatcggcgaaaccgtgcagtgcctgagcgaaagcaccatcggttggcacgcgccgccgaacccgcactctctgggtatcgaaatctgcgcggatggcggtagccacgcgtctttccgtgttccgggccacgcgtacacccgtgaacagtggctggacccgcgtgtttggccggcggttgagcgtgcggcgatcatgtgccgtcagctgtgcgacaaacacggtgttccgaaacgcaaactgtctgttgcggatctgaaggctggccgccgtggcatctgcggtcacaccgatgtaaccgatgcttggcatcagtctgatcatgatgatccaggtccgtggttcccgtgggataaatttatggcggttgttaacggtcatggtggtggttcaagctctgaagaactgactgtggctgatgttaaagcactgcacaatcagattaaacagttaagcgcacaactgagcggttctgttaataaactgcatcacgatgttggtgttgttcaggttcagaacggtgatctgagcaaacgtgttgatgctctgtcctgggttaaaaatccggttaccggtaaactgtggcgtactaaagacgcgctgtggagtgtttggtattacgttctggaatgtcgttctcgtattgatcgtctggaaagtgcggttaacggtctgaaaaaataa

SEQ ID NO: 3 - protein, 108 aa, synthetic construct
FMAVVNGHGGGSSSEELTVADVKALHNQIKQLSAQLSGSVNKLHHDVGVVQVQNGDLSKRVDALSWVKNPVTGKLWRTKDALWSVWYYVLECRSRIDRLESAVNGLKK

SEQ ID NO: 4 - DNA, 347 bp, synthetic construct
  source 1..347: mol_type = other DNA
cgtggttcccgtgggataaattcatggcggttgttaacggtcacggtggtggttcttcttctgaagaactgaccgttgctgatgttaaagcgctgcacaaccagatcaaacagctgtctgcgcagctgagcggttctgttaacaaactgcaccacgatgttggcgttgttcaggttcagaacggtgatctgagcaaacgtgttgatgcgctgtcttgggttaaaaacccggttaccggtaaactgtggcgtaccaaagatgctctgtggtctgtttggtactatgttctggaatgccgtagccgtattgatcgtctggaaagcgcggttaacggtctgaaaaaataa

SEQ ID NO: 5 - protein, 101 aa, synthetic construct
MGGGSSSEELTVADVKALHNQIKQLSAQLSGSVNKLHHDVGVVQVQNGDLSKRVDALSWVKNPVTGKLWRTKDALWSVWYYVLECRSRIDRLESAVNGLKK

SEQ ID NO: 6 - DNA, 306 bp, synthetic construct
  source 1..306: mol_type = other DNA
atgggtggtggttcaagctctgaagaactgactgtggctgatgttaaagcactgcacaatcagattaaacagttaagcgcacaactgagcggttctgttaataaactgcatcacgatgttggtgttgttcaggttcagaacggtgatctgagcaaacgtgttgatgctctgtcctgggttaaaaatccggttaccggtaaactgtggcgtactaaagacgcgctgtggagtgtttggtattacgttctggaatgtcgttctcgtattgatcgtctggaaagtgcggttaacggtctgaaaaaataa

SEQ ID NO: 7 - protein, 89 aa, Propionibacterium freudenreichii
MGGAASPMYADRLDGHQEYPETALFSLQQRLGARFGELSGKIDGLAEALKQVQGGNGVDADAIRAAAAEGAKAGVQAMIDTATVNLEAK

SEQ ID NO: 8 - DNA, 270 bp, Propionibacterium freudenreichii
  source 1..270: mol_type = genomic DNA
atgggtggtgctgcttctccgatgtatgctgatcgtctggatggtcatcaggaatatccggaaaccgctctgttttctctgcagcagcgtctgggcgcgcgtttcggtgaactgagcggcaaaatcgacggcctggcggaagcgctgaaacaggtgcagggcggcaacggcgttgatgcggatgcgatccgtgcggcggcggcggaaggtgcgaaagcgggcgttcaggcgatgatcgataccgcgaccgttaacctggaagcgaaataa